(19) United States
(12) Patent Application Publication
(10) Pub. No.: US 2007/0249062 A1
(43) Pub. Date: Oct. 25, 2007

(54) Microfluidic Chip

(75) Inventor: Yasuhisa Kageyama, Kyoto-shi (JP)

Correspondence Address: FISH & RICHARDSON P.C., P.O. BOX 1022, MINNEAPOLIS, MN, 55440-1022, US

(73) Assignee: ROHM Co., Ltd., Kyoto (JP)

(21) Appl. No.: 11/736,708

(22) Filed: Apr. 18, 2007

(30) Foreign Application Priority Data
Apr. 19, 2006 (JP) 2006-115637 (P)

(51) Int. Cl. G01N 33/558

(52) U.S. Cl. 436/514

(57) ABSTRACT

A microfluidic chip used in latex agglutination for analyzing a small amount of liquid specimen with a latex reagent is provided, in order to suppress adsorption of latex particles into an inner wall surface of a latex reagent retaining chamber of the microfluidic chip, and to improve measuring precision of the latex agglutination. The microfluidic chip includes a retaining chamber for the latex reagent, wherein an absolute value of a surface zeta potential in at least one inner wall surface of the retaining chamber is at least 20 mV.

### BACKGROUND OF THE INVENTION

1. Field of the Invention

The present invention relates to a biochip for examining living samples such as DNA, protein, cells, blood or the like, and to a microfluidic chip that is useful, for example as μ TAS (Micro Total Analysis System) used for chemical synthesis, analysis or the like.

2. Description of the Background Art

A microfluidic chip allows a series of experimental operations carried out in a laboratory to be implemented in one chip measuring about 2 cm square. Therefore, it has many advantages such as follows: samples and reagents are required only in a small amount; costs are low; reaction speeds are fast; high-throughput tests can be performed; and results can immediately be obtained at the location where the sample was taken.

A plan view of a microfluidic chip is shown in “All electronic appliance and machinery companies compete for the biochip market”, Nikkei Biobusiness, Dec. 2003, pp.42-43. This chip is for hepatic function tests. As shown in FIG. 1, blood of about 10 μL is injected from a sample injection inlet 1 into the chip. After the blood is separated into blood cells and blood plasma by centrifugation, only the blood plasma is transferred to a blood plasma retaining chamber 2. In a weighing chamber 3, the amount of the blood plasma is measured, and the blood plasma is transferred to a blending chamber 7. Next, a reagent in a reagent retaining chamber 4 is blended. After the blood plasma and the reagent are mixed in a mixing chamber 5, hepatic function is tested in a measuring chamber 6. The measurement is carried out by irradiating with a short wavelength laser and measuring the light absorption amount with a photodiode. In this manner, a series of operations from the preprocessing of the sampled blood to the measurement thereof can be carried out in a 2 cm-square-chip, to test on the items such as γ-GTP, AST (GOT), ALT (GPT), and lactate dehydrogenase (LDH). The structure of the reagent retaining chamber is illustrated in FIGS. 2A and 2B. FIG. 2A is a perspective view and FIG. 2B is a plan view. The reagent retaining chamber shown in FIGS. 2A and 2B has a depth of 1.5 mm, a radius (r) of 2.9 mm, a flow channel width (L) of 0.3 mm, and a retaining room capacity of 40 μL. Into such a retaining chamber, a latex reagent or the like is injected from reagent injection inlet 28.

A manufacturing method of a microfluidic chip is shown in “All electronic appliance and machinery companies compete for the biochip market”, Nikkei Biobusiness, Dec. 2003, pp.42-43. As shown in FIGS. 4A-4K, according to the method, fine flow channels, the reagent retaining chamber and the like in the microfluidic chip are formed by microfabrication technique in which photolithography, etching and molding are combined. First, a silicon substrate is heated under an oxygen atmosphere to form an SiO₂ film 42 is formed on silicon substrate 41 (FIG. 4A). Thereafter, a resist 43 is formed on SiO₂ film 42 (FIG. 4B). As the resist, resin containing polymethacrylic acid ester such as polymethyl methacrylate (P MA) as its main composition, or a chemically amplified resin having sensitivity to ultraviolet (UV).

Thereafter, a mask 44 is arranged on resist 43. Irradiation of UV 45 through mask 44 is performed (FIG. 4C). Mask 44 is constituted of an UV absorbing layer 44bformed in accordance with the arrangement and shape of the flow channels and the retaining chamber in the manufactured microfluidic chip, and a translucent base 44a. A quartz glass or the like is employed as translucent base 44a, and chrome or the like is employed as absorbing layer 44b. When a positive resist is used, irradiation of UV 45 exposes and change the property of resist 43bonly owing to absorption layer 44b. Therefore, resist 43bis removed by development to leave resist 43a(FIG. 4D). On the other hand, when a negative resist is used, conversely the exposed portion is left and the unexposed portion is removed and, therefore, a mask pattern that is the inverse of that of the positive resist is used.

Next, after plasma etching or wet etching is performed using resist 43aas a mask (FIG. 4E), resist 43ais removed (FIG. 4F). On the obtained SiO₂ film 42a, a metal film is formed by deposition or the like (FIG. 4G). Silicon substrate 41 and SiO_{2 film 42}aare removed by wet etching or mechanical peeling, whereby a mold 46 is obtained (FIG. 4H). Subsequently, using the obtained mold 46, injection molding or the like is carried out with a liquid of molten resin (FIG. 41), whereby a resin-made compact 47ais manufactured (FIG. 4J). As the resin material, thermoplastic resin such as polyethylene terephthalate, polystyrene, polycarbonate, polyvinyl chloride, PMMA are used. Finally, a opposing resin plate 47bis joined, whereby microfluidic chip 47 having the fine channels and the reagent retaining chamber is obtained (FIG. 4K).

One of the methods of analyzing a small amount of component in a liquid specimen is the latex agglutination, which is used for testing blood or the like. In the latex agglutination, as shown in FIG. 3, a latex reagent constituted of latex particles having antibody fixed to the surface is used. The latex particles being mixed with a specimen react with a specific antigen in the specimen. By the antigen-antibody reaction, latex clumps are formed. Accordingly, the antigen amount can be measured based on the change in the turbidity of the reaction liquid. While FIG. 3 illustrates the manner of measuring the antigen amount in the specimen by the latex particles having antibody fixed to the surface, it is possible to similarly measure the antibody amount in the specimen by latex particles having antigens fixed thereto. Exemplary antigen-antibody may be zonisamide-antizonisamide mouse monoclonal antibody, ethosuximide-antiethoxuximide mouse monoclonal antibody and the like.

### SUMMARY OF THE INVENTION

When the latex agglutination is carried out using a microfluidic chip, if the latex reagent is stored for about 10 days in the retaining chamber of the chip, the latex particles adsorb into the entire surface of the inner wall. This reduces the latex concentration in the latex reagent, causing an error in the measurement. Accordingly, a microfluidic chip is provided, which is capable of suppressing adsorption of the latex particles into the inner wall surface of the latex reagent retaining chamber of the microfluidic chip, and which has a high measuring precision of the latex agglutination.

The present invention provides a microfluidic chip used in latex agglutination for analyzing a small amount of liquid specimen with a latex reagent. The microfluidic chip includes a retaining chamber for the latex reagent, characterized in that an absolute value of a surface zeta potential in at least one inner wall surface of the retaining chamber is at least 20 mV, preferably at least 30 mV. Suitably, the inner wall surface of the latex reagent retaining chamber has an arithmetical mean surface roughness Ra of at most 1.0 μm. Desirably, the inner wall surface has a coating layer of fluorine resin or silicone resin.

When the latex agglutination is carried out using this microfluidic chip, the adsorption of the latex particles into the inner wall surface of the latex reagent retaining chamber is suppressed, and the measurement precision is improved. Accordingly, it is useful as a biochip or a microfluidic chip for μTAS.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a plan view of a conventional microfluidic chip.

FIGS. 2A and 2B are schematic views showing the structure of a conventional reagent retaining chamber.

FIG. 3 shows an antigen-antibody reaction in the conventional latex agglutination.

FIGS. 4A-4K are process drawings showing a manufacturing method of a conventional microfluidic chip.

FIG. 5 is a drawing related to the description of a surface zeta potential in an inner wall surface of a latex reagent retaining chamber.

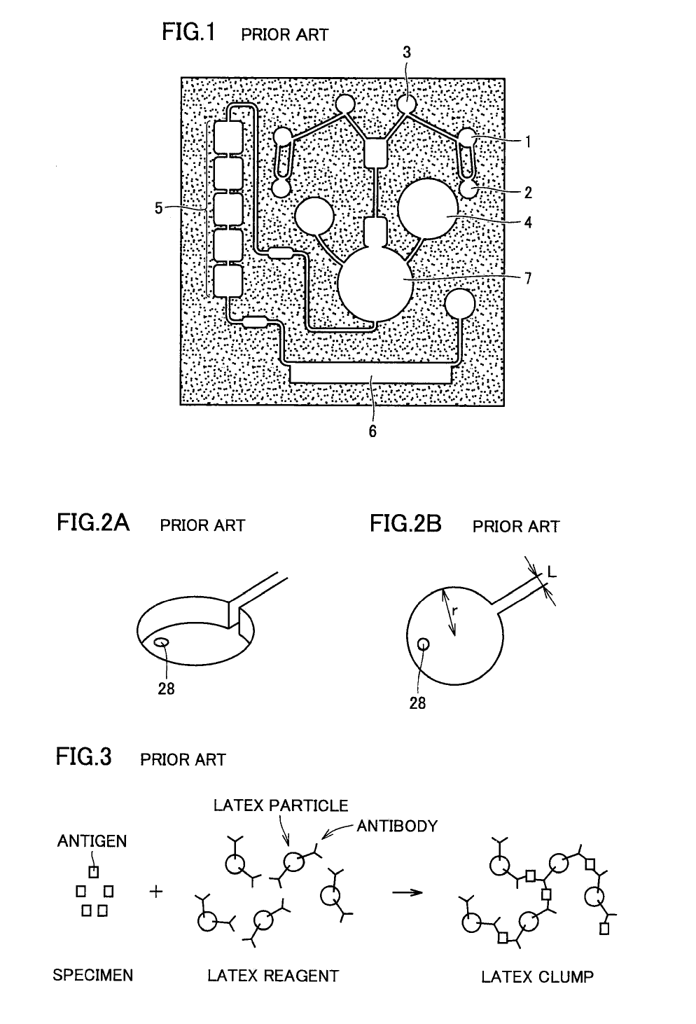

### DESCRIPTION OF THE PREFERRED EMBODIMENTS

The present invention provides a microfluidic chip used in latex agglutination for analyzing a small amount of liquid specimen with a latex reagent. The microfluidic chip includes a retaining chamber for the latex reagent, characterized in that an absolute value of a surface zeta potential in at least one inner wall surface of the retaining chamber is at least 20 mV. By defining the absolute value of the surface zeta potential in the inner wall surface of the latex reagent retaining chamber to be at least 20 mV, adsorption of the latex particles into the inner wall surface can be suppressed. This can prevent reduction in the latex concentration of the latex reagent even if the latex reagent is stored for a long period. Accordingly, errors in the turbidity measurement by the latex agglutination can be reduced to achieve measurement of high precision. From such a standpoint, the absolute value of the surface zeta potential in the inner wall surface of the latex reagent retaining chamber is preferably at least 30 mV, and more preferably at least 35 mV.

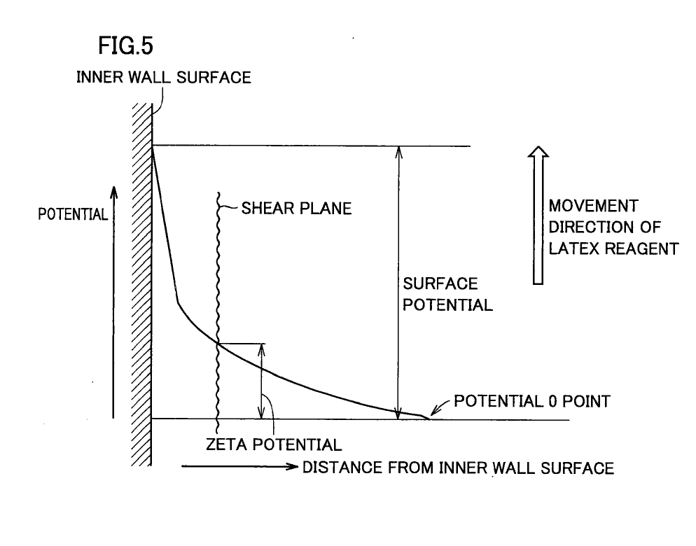

The surface zeta potential means, a potential difference between a point that is sufficiently distanced from the inner wall surface and where potential is zero and a shear plane, when a diffuse electric double layer is formed at the inner wall surface of the latex reagent retaining chamber, as shown in FIG. 5. As used herein, the shear plane means the outermost plane of the diffuse electric double layer that moves along with the movement of a solid body (the latex particulate, the reagent retaining chamber inner wall). As shown in FIG. 5, the surface zeta potential is different from the surface potential immediately above the inner wall surface. The surface zeta potential can be obtained by carrying out electrophoretic light scattering measurement using the laser zata electrometer ELS-8000 available from Otsuka Electronics Co., Ltd., and, with the obtained electric mobility, calculating using the Smoluchowski equation.

Smoluchowski Equation: U=εξ4πη (U: electric mobility, ε: permittivity of solvent, ξ: zeta potential, η: viscosity of solvent)

As to the inner surface wall of the latex reagent retaining chamber, considering that the absolute value of the surface zeta potential is at least 20 mV, preferably the arithmetical mean surface roughness Ra is at most 1.0 μm and more preferably at most 0.5 μm. Measurement of arithmetical mean surface roughness Ra is carried out based on JIS-B0601 and JIS-B0651, and for example by VL2000D available from Lasertec Corporation. Such smoothness of the inner wall surface can be obtained by, for example, molding such as injection molding using a mirror-finished or electropolished mold to have an arithmetical mean surface roughness Ra of at most 0.5 μm.

As to the inner surface wall of the latex reagent retaining chamber, considering that the absolute value of the surface zeta potential is at least 20 mV, preferably a coating layer of fluorine resin or silicone resin is provided. Employing fluorine resin or silicone resin as the coating layer, the absolute value of the surface zeta potential can effectively be controlled. As the fluorine resin, PTFE (polytetrafluoroethylene), PFA (tetrafluoroethylene-perfluoroalkyl vinyl ether copolymer), FEP (tetrafluoroethylene-hexafluoropropylene copolymer), ETFE (ethylene-tetrafluoroethylene copolymer), PVDF (polyvinylidene fluoride), PCTFE (polychlorotrifluoroethylene) or the like can preferably be used. Further, a coating film formed of perfluoroalkyl group containing polymer is also effective. For example, Unidyne available from Daikin Industries, Ltd. is a copolymer of perfluoroalkyl ethyl acrylate, alkyl acrylate, vinyl chloride, and cross-linking monomer. When this copolymer is coated on the inner wall surface of the latex reagent retaining chamber, perfluoroalkyl group covers the surface of the coating film. Alkyl acrylate shows the film forming function, while vinyl chloride and cross-linking monomer provide durability of the coating film and its adhesion to the inner wall surface.

On the other hand, since silicone resin contains trifunctional units (organo silsesquioxane; RSiO _{1.5}) and tetrafinctional units (silicate; SiO2) in a molecule, the three-dimensional network is dense. Therefore, stiff coating and compact can be obtained. Silicone resin includes pure silicone resin and modified silicone resin. Pure silicone resin is constituted of a combination of biftinctional units (diorganosiloxane; R₂SiO) and trifunctional units, or solely of trifunctional units. Pure silicone is basically obtained by hydrolysis of a combination of dichlorosilane and trichlorosilane to increase the molecular weight. After a coating is formed, cross-linkage is formed and insolvability is attained. On the other hand, modified silicone resin includes silicone-modified alkyl resin, silicone-modified epoxy resin, silicone-modified polyester resin, silicone-modified acrylic resin, silicone-modified urethane resin and the like. For example, silicone-modified polyester resin is synthesized by a condensation reaction of a silicone resin intermediate having an alkoxysilyl group and a silanol group and hydroxyl group-containing polyester resin. Silicone-modified alkyl resin is synthesized by a silicone intermediate and alkyl resin of oxidation polymerization type.

### EXAMPLE 1

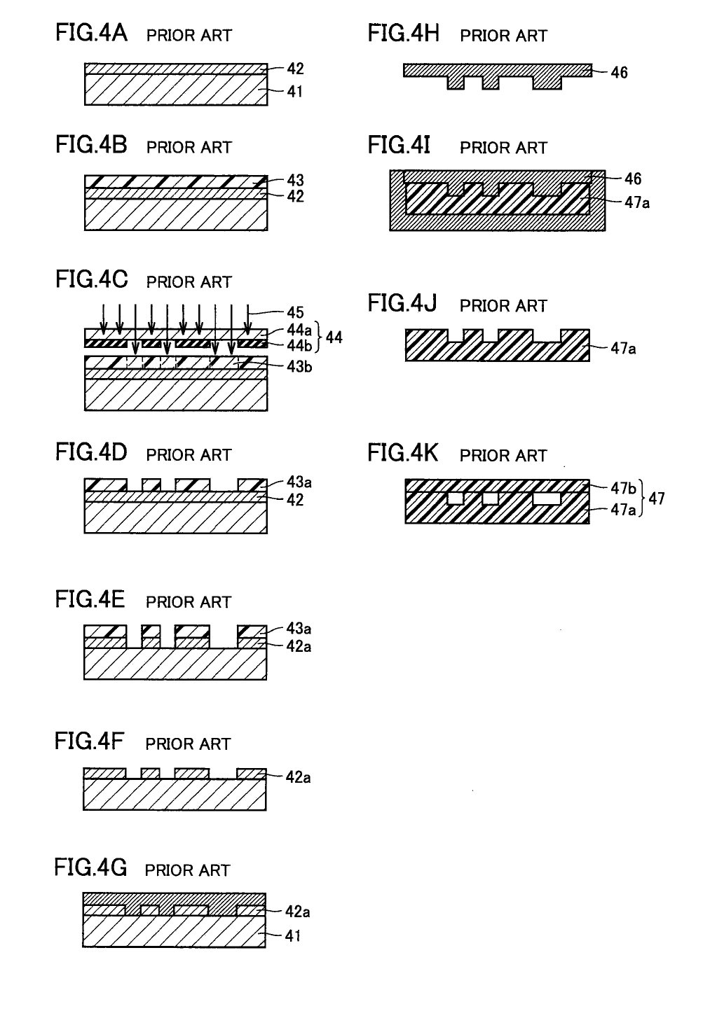

A microfluidic chip was manufactured according to the method described above referring to FIGS. 4A-4K. That is, after SiO₂ film 42 was formed on silicon substrate 41 (FIG. 4A), resist 43 was formed on SiO₂ film 42 (FIG. 4B). As the resist, polymethyl methacrylate (PMMA) was used. Thereafter, a mask 44 was arranged on resist 43. Irradiation of UV 45 through mask 44 was performed (FIG. 4C), and development was carried out (FIG. 4D). Next, after plasma etching was performed (FIG. 4E), resist 43awas removed (FIG. 4F). A metal film was formed by deposition (FIG. 4G). Subsequently, silicon substrate 41 and Sio₂ film 42awere removed by wet etching, whereby a mold 46 was obtained (FIG. 4H).

Thereafter, the molding surface of mold 46 was mirror-finished to attain an arithmetical mean surface roughness Ra of 0.5 μm. Using mold 46, injection molding was carried out with a liquid of molten polystyrene (FIG. 4I), whereby a resin-made compact 47awas obtained (FIG. 4J). The resin-made compact had a latex reagent retaining chamber as shown in FIGS. 2A and 2B, the inner wall surface of which had an arithmetical mean surface roughness Ra of 0.5 μm.

Next, the surface zeta potential in the inner wall surface of the latex reagent retaining chamber was measured. With the inner wall surface being closely in contact with a cell for a flat plate sample, monitor particles to be electrophoresed were injected into the cell. As the monitor particles, a 1.0 mg/mL aqueous dispersion liquid of polystyrene latex particles having a grain size of 234 nm was used. Subsequently, electrophoretic light scattering measurement of the monitor particles was carried out using the laser zata electrometer ELS-8000 available from Otsuka Electronics Co., Ltd., and, with the obtained electric mobility, the zeta potential was calculated. As the cell for a flat plate sample, a flat plate coated with polyacrylamide was used for suppressing the effect of the charges on the cell surface. As a result of the measurement, the absolute value of the surface zeta potential in the inner wall surface of the latex reagent retaining chamber was 39 mV.

Finally, opposing polystyrene resin plate 47bof which inner wall surface had an arithmetical mean surface roughness Ra of 0.5 μm was joined, whereby microfluidic chip 47 having fine channels and reagent retaining chambers was obtained (FIG. 4K). A liquid specimen containing ethosuximide was injected into the obtained microfluidic chip, and a buffer and a latex reagent were respectively injected into the two reagent retaining chambers. Thereafter, the latex agglutination was carried out and the specimen was analyzed. Specifically, the specimen (1.0 μL) and a buffer (17 μL) were mixed and held for five minutes at 37 ° C. Thereafter, the latex reagent (17 μL) were mixed therewith and held for one minute at 37° C. The light absorbance immediately thereafter was measured as light absorbance I. Subsequently, the light absorbance immediately after being held for five minutes at 37 ° C. was measured as light absorbance II. The light absorbance was measured using 7170 available from Hitachi, Ltd. The used latex reagent was formed of latex particles having antiethoxuximide mouse monoclonal antibody fixed to the surface.

After 31 days from the first measurement, the similar operations were carried out again to measure light absorbance I and light absorbance II. Thereafter, the latex reagent retaining chamber was disassembled to visually observe the inner wall surface. As a result, adsorption of the latex particles was not found. The state of the inner wall surface and the measurement result are shown in Table 1. In Table 1, the zata potentials are expressed in absolute values. ΔA means light absorbance II—light absorbance I. ΔA₁ is ΔA at the first measurement. ΔA₂ is ΔA after 31 days.

TABLE 1

| | state of inner wall surface | | | | measurement result | | | | |
|---|---|---|---|---|---|---|---|---|---|
| | Ra (μm) | | coating layer | | zata | first | after 31 days | | |
| | | opposing | | thickness | potential | measurement | | visual | |
| | compact | flat plate | material | (μm) | (mV) | ΔA₁ | ΔA₂ | observation result | (ΔA₂/ΔA₁) × 100% |
| Example 1 | 0.5 | 0.5 | none | none | 39 | 0.3425 | 0.3416 | no adsorption | 99.7 |
| Example 2 | 1.0 | 1.0 | none | none | 50 | 0.3425 | 0.3391 | no adsorption | 99.0 |
| Example 3 | 1.5 | 1.5 | none | none | 62 | 0.3425 | 0.3448 | no adsorption | 100.6 |
| Comparative | 5.0 | 5.0 | none | none | 10 | 0.3425 | 0.2802 | adsorption found | 81.8 |
| Example 1 | | | | | | | | | |
| Comparative | 10.0 | 10.0 | none | none | 5 | 0.3425 | 0.2779 | adsorption found | 81.1 |
| Example 2 | | | | | | | | | |
| Example 4 | 8.0 | 7.0 | fluorine resin | 0.4 | 23 | 0.3425 | 0.3474 | no adsorption | 101.4 |
| Example 5 | 7.0 | 8.0 | fluorine resin | 0.7 | 22 | 0.3425 | 0.3362 | no adsorption | 98.1 |
| Example 6 | 0.8 | 0.7 | fluorine resin | 0.5 | 25 | 0.3425 | 0.3397 | no adsorption | 99.1 |

### Examples 2 and 3

Microfluidic chips were manufactured and measured similarly as in Example 1, except that resin compacts were manufactured respectively using a mold having its molding surface mirror-finished to attain an arithmetical mean surface roughness Ra of 1.0 μm (Example 2) and a mold having its molding surface mirror-finished to attain arithmetical mean surface roughness Ra of 1.5 μm (Example 3), each joined with an opposing resin plate having its inner wall surface similarly mirror-finished. The state of the inner wall surface and the measurement result are shown in Table 1.

### Comparative Examples 1 and 2

Microfluidic chips were manufactured and measured similarly as in Example 1, except that resin compacts were manufactured respectively using a mold of which molding surface had an arithmetical mean surface roughness Ra of 5.0 μm (Comparative Example 1) and a mold of which molding surface had an arithmetical mean surface roughness Ra of 10.0 μm (Comparative Example 2), each joined with an opposing resin plate. The state of the inner wall surface and the measurement result are shown in Table 1.

As can be seen from the result in Table 1, in the inner wall surface of the latex reagent retaining chamber, the absolute value of the surface zeta potential was at least 20 mV when Ra was at most 1.5 μm, even without a coating layer. The adsorption of the latex particles was not found in the inner wall surface of the retaining chamber even after 31 days, and the change in light absorbance (ΔA₂) was the same as the first measurement value (ΔA₁).

### Example 4

Using a mold not having its molding surface mirror-finished, injection molding was carried out to obtain a polystyrene compact. Next, the compact and an opposing resin plate was immersed in a fluorine resin solution. As the fluorine resin, PTFE was employed. Immersion was carried out such that they were entirely immersed in the solution, with the inner wall surface of the compact and opposing resin plate being perpendicular to the surface of the resin solution. Thereafter, they were pulled out as they were. Thereafter, with the inner wall surface being horizontally held, they were dried for three hours at room temperature. The thickness of the PTFE coating layer was 0.4 μm. After the coating layer was formed, arithmetical mean surface roughness Ra of the inner wall surface was measured. Except for the foregoing points, a microfluidic chip was manufactured and measured similarly as in Example 1. The state of the inner wall surface and the measurement result are shown in Table 1.

### Example 5

A microfluidic chip was manufactured and measured similarly as in Example 4, except that a fluorine resin solution prepared in a high concentration was used. The thickness of the PTFE coating layer was 0.7 μm. The state of the inner wall surface and the measurement result are shown in Table 1.

As can be seen from the result in Table 1, in the inner wall surface of the latex reagent retaining chamber, owing to the formation of the PTFE coating layer, the absolute value of the surface zeta potential was at least 20 mV when Ra was at least 1.0 μm. The adsorption of the latex particles was not found in the inner wall surface of the retaining chamber even after 31 days, and the change in light absorbance (ΔA₂) was the same as the first measurement value (ΔA₁).

### Example 6

A microfluidic chip was manufactured and measured similarly as in Example 4, except that a mold having its molding surface mirror-finished was used to carry out injection molding to obtain a resin compact, and that an opposing resin plate having its inner wall surface mirror-finished was used. The state of the inner wall surface and the measurement result are shown in Table 1.

As can be seen from the result in Table 1, forming the coating layer of fluorine resin in the inner wall surface of the latex reagent retaining chamber, with surface roughness Ra being at most 1.0 μm, the adsorption of the latex particles was not found in the inner wall surface of the retaining chamber even after 31 days, and the change in light absorbance (ΔA₂) was the same as the first measurement value (ΔA₁).

Although the present invention has been described and illustrated in detail, it is clearly understood that the same is by way of illustration and example only and is not to be taken by way of limitation, the spirit and scope of the present invention being limited only by the terms of the appended claims.

What is claimed is:

1. A microfluidic chip used in latex agglutination for analyzing a small amount of liquid specimen with a latex reagent, comprising
a retaining chamber for said latex reagent, wherein
an absolute value of a surface zeta potential in at least one inner wall surface of said retaining chamber is at least 20 mV.

2. The microfluidic chip according to claim 1, wherein said absolute value of said surface zeta potential is at least 30 mV.

3. The microfluidic chip according to claim 1, wherein said inner wall surface has an arithmetical mean surface roughness Ra of at most 1.0 μm.

4. The microfluidic chip according to claim 1, wherein said inner wall surface has a coating layer of fluorine resin or silicone resin.
